(19) United States
(12) Patent Application Publication
(10) Pub. No.: US 2007/0142447 A1
(43) Pub. Date: Jun. 21, 2007

(54) Veterinary composition comprising an arylpyrazole and a nitroenamine with enhanced antiparasitic activity

(75) Inventors: Christina Mertens, Boxmeer (NL); Heike Williams, Schwabenheim an der Selz (DE)

Correspondence Address: INTERVET INC.;PATENT DEPARTMENT, PO BOX 318, MILLSBORO, DE, 19966-0318, US

(73) Assignee: Intervet International B.V., Boxmeer (NL)

(21) Appl. No.: 11/698,683

(22) Filed: Jan. 26, 2007

Related U.S. Application Data
(63) Continuation of application No. PCT/EP05/53667, filed on Jul. 27, 2005.

(30) Foreign Application Priority Data
Jul. 28, 2004 (EP) 04103616.1

(51) Int. Cl. A61K 31/4196; A61K 31/4178; A61K 31/137

(52) U.S. Cl. 514/381; 514/383; 514/649

(57) ABSTRACT

The invention relates to antiparasitic combinations of arylpyrazoles and nitroenamines, and their use for controlling insect and acarid infestations on animals.

### CROSS-REFERENCE TO RELATED PATENT APPLICATIONS

This patent claims priority under 35 U.S.C. §120 as a continuation of Int'l Patent Appl. No. PCT/EP2005/053667 (filed Jul. 27, 2005; and published as under Int'l Publ. No. WO 2006/010767 on Feb. 2, 2006), which, in turn, claims priority to European Patent Appl. No. EP04103616.1 (filed Jul. 28, 2004). The entire text of each of the above-referenced patent applications is incorporated by reference into this patent.

### FIELD OF THE INVENTION

The present invention relates to combinations of an arylpyrazole and a nitroenamine; kits comprising such combinations; use of such combinations and kits to control parasitic insect and/or acarid infestations on animals; and use of such combinations and kits for making veterinary medicaments to control parasitic insect and/or acarid infestations on animals.

### BACKGROUND OF THE INVENTION

A number of pests and parasites can infest or infect domestic animals, such as cattle, horses, pigs, sheep, and companion animals (e.g., cats and dogs). These pests and parasites are a nuisance to both the animals and their owners. External parasites, such as ticks, mites, lice, and fleas, irritate the animals and can cause disease, either by themselves, or by carrying vector-transmitted pathogens.

Ticks are important blood-feeding arthropod parasites that, together with mites, belong to the order Acarina. Ticks produce injury after infestation in three respects: direct damage caused by parasitism, such as local injury and blood loss; damage caused by toxins injected by the parasites; and damage caused by the transmission of diseases. Especially in companion animals, ticks can be a source of transmitted diseases.

Fleas (Ctenocephalides felis and Ctenocephalides canis) are the most common cat and dog ectoparasites. Flea infestation of dogs and cats has unpleasant consequences for an animal and its owner. Such infestation, for example, leads to local irritation or troublesome itching, and often results in intense scratching. A large number of animals become allergic to flea saliva, causing itchy local reactions at the sites of flea bites, which, in turn, often leads to lesions and secondary infection due to scratching. Furthermore, flea- and tick-infested animals are constantly exposed to the risk of infestation by parasite-transmitted pathogens, such as Rickettsia, Protozoa, Taenia, or Dipylidium (i.e., a tapeworm transmitted by fleas).

Safe and effective ways to eliminate these parasites are desired for the comfort and well-being of companion animals, the comfort and well-being and comfort of their human associates, and prevention of livestock losses. Thus, there continues to be a need for compounds (and combinations thereof) that: (a) can be used as active agents against parasites, especially parasites that afflict companion animals, such as fleas and ticks; and (b) are effective at low application rates, are selective in biological action, and have low toxicity.

EP0412849 discusses various aryl-1,2,3-triazoles and arylpyrazoles wherein an imidazol(in)e group is attached directly or indirectly at its 2-position to a triazole or pyrazole ring. EP0412849 reports that such compounds exhibit pesticidal activity, and that some such compounds exhibit systemic activity against ectoparasitic insects when administered orally to an animal.

EP0302389 discusses various nitroenamines, and reports that such compounds exhibit activity as contact insecticides and contact acaricides. EP0616494 reports that various 1-[N-(halo-3-pyridylmethyl)]-N-methylamino-1-alkylamino-2-nitroethylene derivatives, in particular, exhibit a pronounced activity against fleas. EP0616494 also reports that such compounds may be orally administered. Nitenpyram is a nitroenamine that is commercially available for controlling acute flea infestation in companion animals. It is sold under the trade name CAPSTAR® by Novartis Inc.

Nitroenamines, such as nitenpyram, are rapidly absorbed via the gastro-intestinal tract and distributed in the blood when they are orally administered (e.g., as tablets). They are, however, excreted rapidly via the urine. Thus, the persistence of antiparasitic activity after single administration is rather short. This limited persistence of antiparasitic activity is a major obstacle for the use of a nitroenamine as a single active ingredient in flea control regimens.

There have been unsuccessful attempts to prolong the persistence of antiparasitic activity of such compounds, e.g., increasing the dose. For nitenpyram, a half-life in the blood of 7.7 hours for cats and 2.8 hours for dogs and a mean residence time of 10.2 hours for cats and 4.1 hours for dogs have been reported (Dryden M W, et al, Proceedings of the Annual Meeting of the American Association of Veterinary Parasitology, 44, pp 1-9 (1999)). This is too short to achieve a significant improvement in persistent efficacy by means of increasing the dose. Both a steep rise in blood values after application and a rapid flattening of the curve are observed, without a significant influence on prolonged bioavailability. Recently, it was reported that nitenpyram levels in host blood were not lethal to fleas after 72 hours (Rust M K et al, J. Med. Entomol., 40(5), pp 678-81 (2003)).

Because of the limited persistence of antiparasitic activity, tablets or injections have to be administered at short intervals, preferably every other day, which means the owner is inconvenienced by having to repeatedly administer the treatment or visit the veterinarian. In addition, such an intensive treatment requires a burdensome amount of compliance. This can cause stress to the animal and its owner, which, in turn, can manifest itself as an aversion to the treatment and ultimately a discontinuation of the treatment. Thus, there is a need to prolong the systemic action of nitroenamines against fleas.

### SUMMARY OF THE INVENTION

This invention is, in part, directed to a veterinary composition. The composition comprises:
an arylpyrazole of Formula (I): 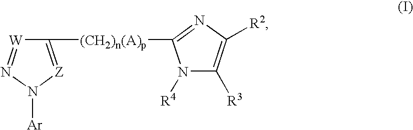
a nitroenamine of Formula (II): 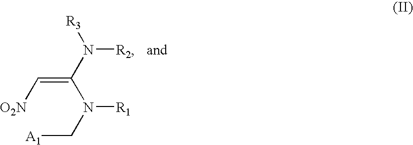
a physiologically acceptable formulation excipient.
   The substituents in Formulas (I) and (II) are defined as follows:

Ar is 2,6-dichloro-4-trifluoromethylphenyl or 2-nitro-4-trifluoromethylphenyl.

A is S(O)ₘ, CH═CH, O, or NH.

As to W and Z:
W is N, and Z is CR⁵; or
W is CR¹, and Z is N or CR⁵.

R¹ is hydrogen, optionally substituted alkyl, halogen, or R²⁰S(O)_{q}.

R² and R³ are hydrogen, optionally substituted alkyl, optionally substituted alkenyl, optionally substituted alkynyl, aryl, cyano, halogen, nitro, YR²⁰, S(O)₂NR⁸R⁹, CHO, NR⁸R⁹, or CYNR⁸R⁹.

R⁴ is hydrogen, optionally substituted alkyl, optionally substituted alkenyl, acyl, or optionally substituted alkoxycarbonyl. R⁴, however, is not alkyl when W is CR¹, Z is CR⁵, and n and p are both zero.

R⁵ is hydrogen, alkyl, optionally substituted amino, or halogen.

R⁸ and R⁹ are:
the same or different, and
hydrogen, optionally substituted alkyl, acyl, or aryl.

R²⁰ is optionally substituted alkyl.

Y is O or S.

m is zero, 1, or 2.

p is zero or 1.

n is zero, 1, or 2.

q is zero, 1, or 2.

R₁ is hydrogen, C₁-C₆ alkyl, or C₃-C₇ cycloalkyl.

R₂ is hydrogen, C₁-C₆ alkyl, or C₃-C₇ cycloalkyl.

R₃ is hydrogen or C₁-C₆ alkyl.

A₁ is heterocyclyl optionally substituted with one or more identical or different halogen.

Unless otherwise stated, any alkyl, alkoxy, or alkylthio has from 1 to 4 carbon atoms.

Unless otherwise stated, any alkenyl or alkynyl has from 2 to 5 carbon atoms.

Unless otherwise stated, any substituted alkyl, alkoxy, alkylthio, alkenyl, or alkynyl is substituted by one or more of the same or different groups selected from the group consisting of halogen, YR²⁰, dihalocyclopropyl, cyano, nitro, optionally substituted amino, acyloxy, and aryl.

Unless otherwise stated, any aryl is phenyl optionally substituted by halogen, alkyl, haloalkyl, alkoxy, haloalkoxy, alkylthio, haloalkylthio, haloalkylsulphonyl, cyano, or nitro.

Unless otherwise stated, any acyl is alkanoyl that has from 1 to 4 carbon atoms, alkylsulphonyl, or haloalkylsulphonyl.

Unless otherwise stated, any optionally substituted amino is NR⁸R⁹.

This invention is, in part, also directed to a method for controlling a parasitic insect and/or acarid infestation. The method comprises administering a compound of Formula (I) and a compound of Formula (II) to an animal.

This invention is, in part, also directed to a kit useful for controlling a parasitic insect and/or acarid infestation. The kit comprises a compound corresponding in structure to Formula (I), a compound corresponding in structure to Formula (II), and instructions for controlling a parasitic insect and acarid infestation on an animal.

### BRIEF DESCRIPTION OF THE DRAWINGS

FIG. 1 shows the percentage efficacy against ticks for dogs with a nitenpyram dosing schedule of 15 mg/kg (Group A), 10 mg/kg (Group B), and 5 mg/kg (Group C). In all instances, 5-chloro-1-(2,6-dichloro-4-trifluoromethylphenyl)-4-(4,5-dicyano-1H-imidazol-2-yl)-3-isopropyl-1H-pyrazole (“Compound 22c”) was administered orally at an initial dose of 4 mg/kg, followed by a maintenance dose of 2 mg/kg in the second treatment week. The initial Compound 22c dose of 4 mg/kg was split into 2×2 mg/kg, and administered on two consecutive days simultaneously with the nitenpyram.

FIG. 2 shows the percentage efficacy against fleas for the dogs of Groups A, B, and C.

### DETAILED DESCRIPTION OF PREFERRED EMBODIMENTS

Surprisingly, Applicants have found that a composition comprising a combination of an arylpyrazole of Formula (I) and a nitroenamine of Formula (II) provides persistent activity against fleas for an extended period of time at a dosage that is used to control ticks. This enhanced kill activity against acarids and fleas provides excellent control of the most important ectoparasites in companion animals. This is rather unexpected because it is known that nitroenamines alone have a short persistence of activity against fleas, as outlined above. And arylpyrazoles alone generally have limited activity against fleas in a dosage used to control ticks.

Arylpyrazoles of Formula (I), as well as methods for their preparation, have been described in, for example, EP0412849.

Nitroenamines of Formula (II), as well as methods for their preparation, have been described in, for example, EP0302389.

The alkyl groups present in the definitions of the substituents may be straight-chain or branched, depending on the number of carbon atoms, and they may be for example methyl, ethyl, propyl, butyl, pentyl, or hexyl, as well as the branched isomers thereof, for example isopropyl, isobutyl, sec.-butyl, tert-butyl, isopentyl, neopentyl, or isohexyl. C₃ to C₇ cycloalkyl is cyclopropyl, cyclobutyl, cyclopentyl, cyclohexyl, or cycloheptyl.

Halogen atoms are, for example, fluorine, chlorine, bromine, or iodine, preferably fluorine or chlorine, especially chlorine. A partially or completely halogenated substituent may contain one or more identical or different halogen atoms.

In the context of the present invention, heterocyclyl is understood to mean aliphatic or aromatic cyclic radicals, which contain at least one oxygen, sulphur, or nitrogen atom. Five- and six-membered heterocycles are preferred. Heterocyclyl typically includes groups such as dioxolanyl, pyrrolidonyl, piperidinyl, morpholinyl, pyridyl, pyrrolyl, pyrryl, furyl, thienyl, imidazolyl, tetrahydrofuryl, tetrahydropyranyl, dihydrofuryl, dihydropyranyl, isoxazoyl, oxazolyl, thiazolyl, oxazolinyl, oxazolidinyl, and imidazolinyl. Preference is given especially to those which are un-substituted or have one or two halogen atoms. Halogen in this case denotes fluorine, chlorine, or bromine, but especially chlorine. Of these heterocyclic radicals, pyridyl, thiazolyl, and tetrahydrofuryl are especially notable. Especially preferred are 5,6-dichloropyridin-3-yl, especially 6-chloropyridin-3-yl, but also 5-chlorothiazol-3-yl and tetrahydrofur-3-yl, and, in particular, 6-chloropyridin-3-yl.

A prominent representative of the nitroenamines of Formula (II) is nitenpyram (INN) of Formula (III): 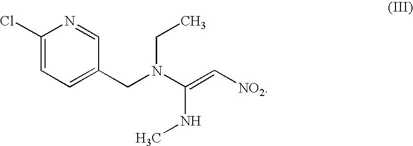
The IUAPC name is (E)-N-(6-chloro-pyridin-3-ylmethyl)-N-ethyl-N-methyl-2-methyl-2-nitrovinylidenediamine. Nitenpyram and its preparation are described in EP0302389, page 63, Example 41.

A prominent representative of an arylpyrazoles of Formula (I) for use in the composition according to the invention is 5-chloro-1-(2,6-dichloro-4-trifluoromethylphenyl)-4-(4,5-dicyano-1H-imidazol-2-yl)-3-isopropyl-1H-pyrazole, which corresponds in structure to Formula (IV): 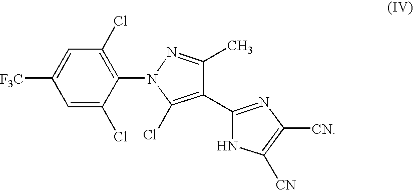
This compound is identified as Compound 22c in EP0412849. The synthesis of this compound is described in Example 22b of EP0412849. In the remainder of this application, this compound is identified as “Compound 22c”.

Another embodiment of this invention relates to the use of the compositions of this invention to manufacture a veterinary medicament for controlling parasitic insect and acarid infestations on animals.

To form the composition according to the invention, the active ingredients may be present in the dosage form as true mixtures, but they also may be administered individually in separate dosage forms and form mixtures only when they are in the host organism.

When the compound of Formula (I) and the compound of Formula (II) are administered individually in separate dosage forms, they are administered in parallel. Parallel means that the active ingredients may be administered at the same time, that is simultaneously, but they may also be administered sequentially, that is one after the other, i.e., so that they are present together for certain periods at the latest in the host organism, so that the desired effect arises.

The active ingredients are preferably administered simultaneously. When given simultaneously, the composition according to the invention is preferably presented as a single dosage form comprising both the compound of Formula (I) and the compound of Formula (II) in a single formulation.

Preferably, the combination of active ingredients according to the invention is administered systemically. “Systemic administration” is the administration of the combination at a site remote from the site where the parasite resides, so that the active compound can be ingested by the feeding parasite along with the blood, body fluids, or tissue, such as skin of the host animal, and then can exhibit activity against the parasite.

In accordance with the present invention, a systemic administration is achieved by several forms. For example, the composition may be administered in an oral formulation or parenterally, e.g., by injection as an implant or bolus, or by a topical administration method, such as pour-on or spot-on.

The compositions of the invention are preferably administered in an oral formulation. The term “oral formulation” means that the active ingredients are formulated into a product suitable for administering to the animal via the mouth. These formulations include, but are not limited to tablets, capsules, liquids, gels, pastes, oral sprays, buccal formulations, powders, granules, chewable treats, or animal feeds containing the active ingredients.

The composition does not necessarily have to be administered to the animal directly. Oral administration includes, for example, the administration of animal food, e.g., for dogs or cats, which contains the composition according to the invention. The composition can be administered in a form that can be dripped onto the food or into the drinking water, or in other forms that can be mixed with the animal food. Such forms include, for example, biscuits, titbits, chews, capsules, or tablets.

Other forms of non-direct oral administration include for example the application of the composition onto the coat of the animal and its later ingestion during the self cleaning of the animal.

Preferably, the compositions according to the invention are provided as tablets, preferably chewable tablets, that can be given to, for example dogs or cats, as a treat.

Conventional tablets generally comprise the active ingredients, a diluent to assist in increasing the powder mass to a convenient size and improve compressibility, a binder to hold the compressed powder together, and a lubricant to assist in densification and ejection from the tablet die. They also may contain a disintegrate to improve disintegration and dissolution, as well as stabilizers, colours, and flavours. Tablets are often coated to improve appearance or taste, or to alter the dissolution properties. Tablets can be designed to dissolve fast or slowly, and depending on the actual volume and compressibility of the drug, they may be large or small. They can be made chewable or to dissolve under the tongue or in the pouch of the cheek. They may contain further additives that stimulate voluntary ingestion by the animal, such as suitable odorous substance, taste substances, scents, or flavorings.

The composition according to the invention also can be administered as a liquid formulation. Conventional liquid formulations for oral administration are usually solutions, suspensions, or emulsions of the active ingredients, together with suitable diluents, solvents, flavors, and colours to form a dosage form.

The compositions according to the current invention conventionally further comprise physiologically acceptable formulation excipients known in the art, such as those described in “Gennaro, Remington: The Science and Practice of Pharmacy” (20th Edition, 2000) (incorporated by reference herein). All such components, carriers, and excipients must be substantially pharmaceutically or veterinary pure, non-toxic in the amounts employed, and compatible with the active ingredients.

The compositions of the invention are intended for use for controlling a parasitic insect and acarid infestation. The term “controlling a parasitic insect and acarid infestation” refers to preventing, reducing, or eliminating an infestation by such parasites on animals, preferably by killing the insects and/or acarids within hours or days.

The term “parasitic insect and acarid” refers to ectoparasites, such as insect and acarine pests that commonly infest or infect animals. Examples of such ectoparasites include the egg, larval, pupal, nymphal, and adult stages of lice, fleas, mosquitoes, mites, ticks biting, or nuisance fly species. Especially important are the adult stages of fleas and ticks.

In general, the composition according to the invention will contain an effective amount of the active ingredients, meaning a non-toxic but sufficient amount to provide the desired control effect. A person skilled in the art using routine experimentation may determine an appropriate “effective” amount in any individual case. Such an amount will depend on the age, condition, weight, and type of the target animal. Tablets may be formulated to contain an amount of active ingredients that is adjusted to animals in a specific weight range.

The animals may receive a monthly, weekly, or daily dosage, optionally preceded by a higher loading dose (initial higher dose). The treatment can, for example, be continuing or seasonal.

Preferably, the treatment is carried out so as to administer to the animal a composition comprising a dose of from 0.1 to 20 mg/kg bodyweight, and, in particular, from 0.5 to 10 mg/kg bodyweight, and, most preferably, from 1 to 5 mg/kg bodyweight of the compound of Formula (I); and a dose of from 0.1 to 50 mg/kg bodyweight, and, in particular, from 1 to 30 mg/kg bodyweight, and, most preferably, from 5 to 15 mg/kg bodyweight of compound of Formula (II) to be administered at weekly interval. These dosages have been proven to be effective, especially in companion animals, such as dogs or cats.

Another aspect of the invention is a kit useful in the treatment of a parasite infestation of insects and/or acarids in an animal, which comprises a compound of Formula (I) and of Formula (II) in a veterinary-acceptable formulation; and instructions for the control of parasitic insect and acarid infestations on animals.

The compositions of the present invention may be prepared in a manner known per se for example by means of conventional mixing, granulating, coating, dissolving, or lyophilising processes.

In general the composition according to the current invention can be administered to all species of animals that have insect or acarid pest infestation. The recipient of the formulation may be a livestock animal (such as sheep, cattle, pig, goat, or poultry), a laboratory test animal (such as a guinea pig, rat, or mouse), or a companion animal (such as a dog, cat, rabbit, ferret, or horse). The compositions according to the invention are especially suitable for use in companion animals, such as dogs, cats, or ferrets.

### EXAMPLE

### Efficacy of Different Dosages of Nitenpyram in Combination with Compound 22c

### Materials and Methods

For all treatment groups, Compound 22c was administered orally at an initial dose of 4 mg/kg body weight (bw), followed by a maintenance dose of 2 mg/kg bw in the second treatment week. The initial dose of 4 mg/kg Compound 22c was split into 2×2 mg/kg bw of Compound 22c applied to dogs on two consecutive days simultaneously with nitenpyram.

For nitenpyram, a dosing schedule of 15 mg/kg bw (Group A), 10 mg/kg bw (Group B), and 5 mg/kg bw (Group C) was used. Group D remained untreated as a control.

Four groups of 3 dogs each were infested with fleas (Ctenocephalides felis) and ticks (Rhipicephalus sanguineus) before and repeatedly at different time points after treatment.

The parasite burden of individual dogs was assessed 48 hours after the first treatment and after each following infestation by removal and counting of ticks and fleas. Ticks and fleas were classified according to vitality (dead/alive).

The details of the study procedures are indicated below:

Study animals

| | |
|---|---|
| Species: | Domestic dog |
| Number: | 16 |
| Breed: | Beagle |
| Body weight: | 11-17 kg |
| Age: | 3-6 years |
| Gender: | male and 1x male, gelded |

### Parasite Infestation

Dogs were experimentally infested with laboratory strains of Rhipicephalus sanguineus (80 unfed adults; sex ratio 1:1) and Ctenocephalides felis (100 adults, similar age; sex ratio approx. 1:1). Fleas and ticks were directly applied onto the back of the animal. The dogs were sedated while distributing the parasites.

Ticks (Rhipicephalus sanguineus) and fleas (Ctenocephalides felis) were infested on day −2, day +3, day +5, day +7, day +10, and day +12.

Product specification
Compound 22c Tablet

| | |
|---|---|
| Active ingredient: | 10 mg Compound 22c Tablet |
| Dosage (per os): | 4 mg/kg bw initial dose (Group A, B C) |
| | 2 mg/kg bw maintenance dose (Group A, B, C) |
| Excipients: | lactose monohydrate, corn starch, pre-gelatinized |
| | starch, silica, colloidal anhydrous, sodium |
| | carboxymethyl-cellulose, magnesium stearate |

Nitenpyram Tablet

| | |
|---|---|
| Commercial product: | CAPSTAR ® (Novartis) |
| Dosage (per os): | 15 mg/kg bw (Group A) |
| | 10 mg/kg bw (Group B) |
| | 5 mg/kg bw (Group C) |

### Treatment

TABLE 1
INFESTATION, TREATMENT, AND ASSESSMENT SCHEME

| | | Dogs | Investigational | | |
|---|---|---|---|---|---|
| | | per | veterinary | | |
| Day | Group | group | product | Dose | Application |
| 0 | A | 3 | Compound 22c | 2 mg/kg BW | Per os |
| | B | | | | |
| | C | | | | |
| +1 | A | 3 | Compound 22c | 2 mg/kg BW | Per os |
| | B | | | | |
| | C | | | | |
| +7 | A | 3 | Compound 22c | 2 mg/kg BW | Per os |
| | B | | | | |
| | C | | | | |
| 0 | A | 3 | Nitenpyram | 15 mg/kg BW | Per os |
| | B | | | 10 mg/kg BW | |
| | C | | | 5 mg/kg BW | |
| +1 | A | 3 | Nitenpyram | 15 mg/kg BW | Per os |
| | B | | | 10 mg/kg BW | |
| | C | | | 5 mg/kg BW | |
| 7 | A | 3 | Nitenpyram | 15 mg/kg BW | Per os |
| | B | | | 10 mg/kg BW | |
| | C | | | 5 mg/kg BW | |

### Evaluation of Tick and Flea Numbers

Parasite assessments on the dogs were made 48 hours after the first treatment. and after each re-infestation on day +2, day +5, day +7, day +9, day +12, and day +14. The flea count on each dog was conducted by combing until no flea was recovered for at least 5 minutes, and the number of live fleas was recorded. Ticks on the dogs were counted by palpation, removed where attached, and classified.

### Calculation of Efficacy

Efficacy calculation was based on the arithmetic means of the number of ticks/fleas on treated dogs compared to that of the control group. For calculation of efficacy (%), the following formula (according to Abbott's formula) is used:
Efficacy=100×(mc−mr)/mc
Control group (mc): mean number of live fleas on the host animals
   mean number of live ticks on the host animals
Treatment group (mr): mean number of live fleas on the host animals
   mean number of live ticks on the host animals

### Results

### Efficacy against Rhipicephalus Sanguineus

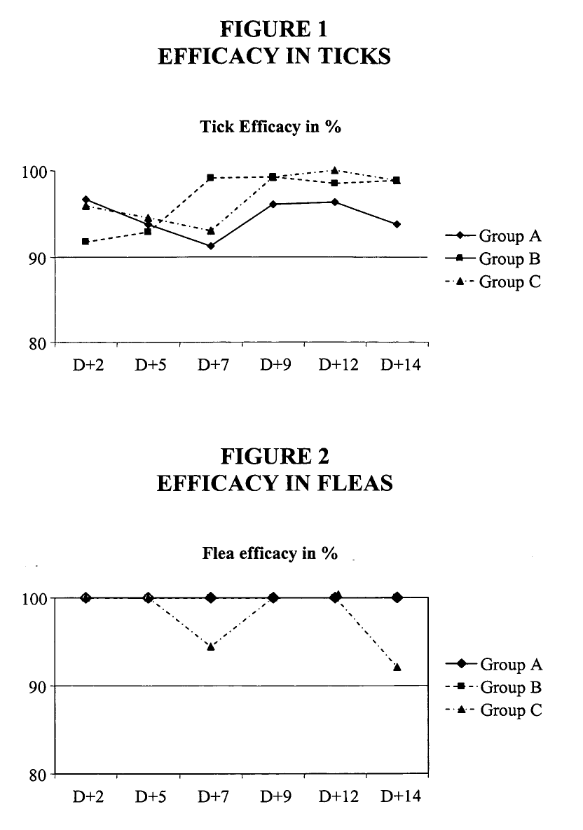

The numbers of ticks and percentage efficacy are given in Table 2. The percentage efficacy against ticks is given in FIG. 1.

TABLE 2
EFFICACY AGAINST RHIPICEPHALUS SANGUINEUS

| | Group A | | Group B | | Group C | |
|---|---|---|---|---|---|---|
| Day after | Mean # | Group A | Mean # of | Group B | Mean # of | Group C |
| treatment | of ticks | Efficacy (%) | ticks | Efficacy (%) | ticks | Efficacy (%) |
| D +2 | 1.33 | 96.7% | 3.33 | 91.7% | 1.67 | 95.9% |
| D +5 | 2.67 | 93.7% | 3.00 | 92.9% | 2.33 | 94.5% |
| D +7 | 3.33 | 91.2% | 0.33 | 99.1% | 2.67 | 93.0% |
| D +9 | 1.67 | 96.1% | 0.33 | 99.2% | 0.33 | 99.2% |
| D +12 | 1.67 | 96.3% | 0.67 | 98.5% | 0.00 | 100% |
| D +14 | 1.67 | 93.8% | 0.33 | 98.8% | 0.33 | 98.8% |

For group A treated with an initial dose of 4 mg/kg bw Compound 22c (split into 2×2 mg/kg bw on two consecutive days) plus 15 mg/kg bw nitenpyram, an immediate tick efficacy of 96.7% was achieved. Activity decreased slightly to 93.7% and 91.2% five and seven days after the first treatment. After re-treatment with 2 mg/kg Compound 22c plus 15 mg/kg bw nitenpyram, in the second week, tick efficacy of 93.8 to 96.3% was demonstrated.

Group B was treated with an initial dose of 4 mg/kg bw Compound 22c (split into 2×2 mg/kg bw on two consecutive days) plus 10 mg/kg bw nitenpyram, and showed an initial efficacy of 91.7% that increased to 92.9% and 99.1% five and seven days after the first treatment. After re-treatment with 2 mg/kg bw Compound 22c plus 10 mg/kg bw nitenpyram, efficacy in the second week was in the range of 98.5 to 99.2%.

For group C treated with an initial dose of 4 mg/kg bw Compound 22c (split into 2×2 mg/kg bw on two consecutive days) plus 5 mg/kg bw nitenpyram, an immediate tick efficacy of 95.9% was achieved. Five and seven days after first treatment, 94.5% and 93.0% efficacies were demonstrated. Re-treatment with 2 mg/kg bw Compound 22c plus 5 mg/kg bw nitenpyram increased efficacy to 98.8 up to 100% in the second week.

Overall, excellent tick efficacy was demonstrated with a combination of Compound 22c and nitenpyram. All treatment groups fulfilled the guideline requirements of at least 90% tick efficacy (“Testing and evaluation of the efficacy of antiparasitic substances for the treatment and prevention of tick and flea infestation in dogs and cats” EMEA/CVMP/005/00).

### Efficacy Against Ctenocephalides Felis

The numbers of fleas and the percentage efficacy against fleas are given in Table 3. The percentage efficacy against fleas is given in FIG. 2.

TABLE 3
EFFICACY AGAINST CTENOCEPHALIDES FELIS

| | Group A | | Group B | | Group C | |
|---|---|---|---|---|---|---|
| Day after | Mean # of | Group A | Mean # of | Group B | Mean # of | Group C |
| treatment | fleas | Efficacy (%) | fleas | Efficacy (%) | fleas | Efficacy (%) |
| D +2 | 0.0 | 100 | 0.0 | 100.0 | 0.0 | 100.0 |
| D +5 | 0.0 | 100 | 0.0 | 100.0 | 0.0 | 100.0 |
| D +7 | 0.0 | 100 | 0.0 | 100.0 | 5.0 | 94.4 |
| D +9 | 0.0 | 100 | 0.0 | 100.0 | 0.0 | 100.0 |
| D +12 | 0.0 | 100 | 0.0 | 100.0 | 0.0 | 100.0 |
| D +14 | 0.0 | 100 | 0.0 | 100.0 | 7.0 | 92.1 |

Group A (15 mg/kg bw nitenpyram) and Group B (10 mg/kg bw nitenpyram) achieved a flea efficacy of 100% for the entire study period.

For Group C (5 mg/kg bw nitenpyram), a therapeutic and prophylactic flea efficacy of 100% was achieved in the first week of study. Seven days after first treatment, efficacy decreased to 94.4%. Three and five days after re-treatment, efficacies were high with 100%. At the end of the second treatment week, flea activity achieved 92.1%.

Overall, the combination of 4 mg/kg bw Compound 22c (split into 2×2 mg/kg bw on two consecutive days) with 15 mg/kg bw and 10 mg/kg bw nitenpyram showed an excellent efficacy against fleas. The lower dosage of 5 mg/kg bw nitenpyram demonstrated a very good efficacy up to 5 days after treatment. 5 mg/kg bw nitenpyram in combination with Compound 22c showed a good efficacy against fleas.

We claim:

1. A veterinary composition, wherein:
the composition comprises:
a compound of Formula (I): 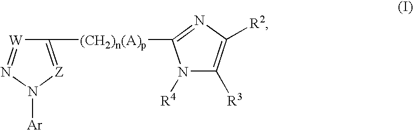
a compound of Formula (II): 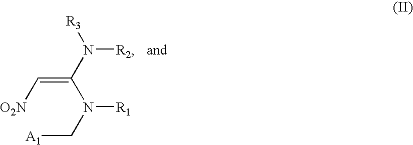
a physiologically acceptable formulation excipient;
Ar is 2,6-dichloro-4-trifluoromethylphenyl or 2-nitro-4-trifluoromethylphenyl;
A is S(O)ₘ, CH═CH, O, or NH;
as to W and Z:
W is N, and Z is CR⁵; or
W is CR¹, and Z is N or CR⁵;
R¹ is hydrogen, optionally substituted alkyl, halogen, or R²⁰S(O)_{q};
R² and R³ are hydrogen, optionally substituted alkyl, optionally substituted alkenyl, optionally substituted alkynyl, aryl, cyano, halogen, nitro, YR²⁰, S(O)₂NR⁸R⁹, CHO, NR⁸R⁹, or CYNR⁸R⁹;
R⁴ is hydrogen, optionally substituted alkyl, optionally substituted alkenyl, acyl, or optionally substituted alkoxycarbonyl, except R⁴ is not alkyl when W is CR¹, Z is CR⁵, and n and p are both zero;
R⁵ is hydrogen, alkyl, optionally substituted amino, or halogen;
R⁸ and R⁹ are:
the same or different, and
are hydrogen, optionally substituted alkyl, acyl, or aryl;
R²⁰ is optionally substituted alkyl;
Y is O or S;
m is zero, 1, or 2;
p is zero or 1;
n is zero, 1, or 2;
q is zero, 1, or 2,
R₁ is hydrogen, C₁-C₆-alkyl, or C₃-C₇-cycloalkyl;
R₂ is hydrogen, C₁-C₆-alkyl, or C₃-C₇-cycloalkyl;
R₃ is hydrogen or C₁-C₆-alkyl;
A₁ is heterocyclyl optionally substituted with one or more identical or different halogens;
unless otherwise stated, any alkyl, alkoxy, and alkylthio has from 1 to 4 carbons;
unless otherwise stated, any alkenyl or alkynyl has from 2 to 5 carbons;
unless otherwise stated, any substituted alkyl, alkoxy, alkylthio, alkenyl, or alkynyl is substituted by one or more of the same or different groups selected from the group consisting of halogen, YR²⁰, dihalocyclopropyl, cyano, nitro, optionally substituted amino, acyloxy, and aryl;
unless otherwise stated, any aryl is phenyl optionally substituted by halogen, alkyl, haloalkyl, alkoxy, haloalkoxy, alkylthio, haloalkylthio, haloalkylsulphonyl, cyano, or nitro;
unless otherwise stated, any acyl is alkanoyl having from 1 to 4 carbons, alkylsulphonyl, or haloalkylsulphonyl; and
unless otherwise stated, any optionally substituted amino corresponds to NR⁸R⁹.

2. The composition according to claim 1, wherein the compound of Formula (I) is 5-chloro-1-(2,6-dichloro-4-trifluoromethylphenyl)-4-(4,5-dicyano-1H-imidazol-2-yl)-3-methyl-1-H-pyrazole.

3. The composition according to claim 2, wherein the compound of Formula (II) is N-(6-chloro-pyridin-3ylmethyl)-N-ethyl-N-methyl-2-methyl-2-nitrovinylidenediamine.

4. The composition according to claim 1, wherein the compound of Formula (II) is N-(6-chloro-pyridin-3ylmethyl)-N-ethyl-N-methyl-2-methyl-2-nitrovinylidenediamine.

5. A method for controlling a parasitic insect and/or acarid infestation, wherein:
the method comprises administering an arylpyrazole and a nitroenamine to an animal;
the arylpyrazole corresponds in structure to Formula (I): 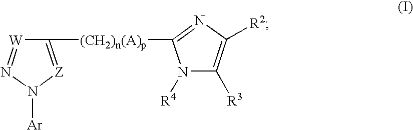
the nitroenamine corresponds in structure to Formula (II): 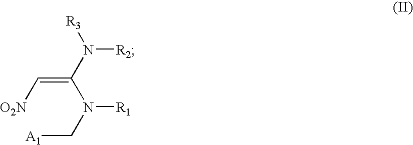
Ar is 2,6-dichloro-4-trifluoromethylphenyl or 2-nitro-4-trifluoromethylphenyl;
A is S(O)ₘ, CH═CH, O, or NH;
as to W and Z:
W is N, and Z is CR⁵; or
W is CR¹, and Z is N or CR⁵;
R¹ is hydrogen, optionally substituted alkyl, halogen, or R²⁰S(O)_{q};
R² and R³ are hydrogen, optionally substituted alkyl, optionally substituted alkenyl, optionally substituted alkynyl, aryl, cyano, halogen, nitro, YR²⁰, S(O)₂NR⁸R⁹, CHO, NR⁸R⁹, or CYNR⁸R⁹;
R⁴ is hydrogen, optionally substituted alkyl, optionally substituted alkenyl, acyl, or optionally substituted alkoxycarbonyl, except R⁴ is not alkyl when W is CR¹, Z is CR⁵, and n and p are both zero;
R⁵ is hydrogen, alkyl, optionally substituted amino, or halogen;
R⁸ and R⁹ are:
the same or different, and
are hydrogen, optionally substituted alkyl, acyl, or aryl;
R²⁰ is optionally substituted alkyl;
Y is O or S;
m is zero, 1, or 2;
p is zero or 1;
n is zero, 1, or 2;
q is zero, 1, or 2;
R₁ is hydrogen, C₁-C₆-alkyl, or C₃-C₇-cycloalkyl;
R₂ is hydrogen, C₁-C₆-alkyl, or C₃-C₇-cycloalkyl;
R₃ is hydrogen or C₁-C₆-alkyl;
A₁ is heterocyclyl optionally substituted with one or more identical or different halogens;
unless otherwise stated, any alkyl, alkoxy, and alkylthio has from 1 to 4 carbons;
unless otherwise stated, any alkenyl or alkynyl group has from 2 to 5 carbons;
unless otherwise stated, any substituted alkyl, alkoxy, alkylthio, alkenyl, or alkynyl is substituted by one or more of the same or different groups selected from the group consisting of halogen, YR²⁰, dihalocyclopropyl, cyano, nitro, optionally substituted amino, acyloxy, and aryl;
unless otherwise stated, any aryl is phenyl optionally substituted by halogen, alkyl, haloalkyl, alkoxy, haloalkoxy, alkylthio, haloalkylthio, haloalkylsulphonyl, cyano, or nitro;
unless otherwise stated, any acyl is alkanoyl having from 1 to 4 carbons, alkylsulphonyl, or haloalkylsulphonyl; and
unless otherwise stated, any optionally substituted amino corresponds to NR⁸R⁹.

6. A method according to claim 5, wherein the arylpyrazole and the nitroenamine are administered together in a single dosage form.

7. A method according to claim 5, wherein:
at least a portion of the arylpyrazole is administered in a first dosage form, and
at least a portion of the nitroenamine is administered in a second dosage form that is separate from the first dosage form.

8. A method according to claim 5, wherein the arylpyrazole and the nitroenamine are administered to the animal systemically.

9. A method according to claim 5, wherein the arylpyrazole and the nitroenamine are orally administered to the animal.

10. A method according to claim 5, wherein:
the arylpyrazole is administered in a dose of from 0.5 to 10 mg/kg bodyweight, and
the nitroenamine is administered in a dose of from 1 to 30 mg/kg bodyweight.

11. A method according to claim 5, wherein the arylpyrazole comprises 5-chloro-1-(2,6-dichloro-4-trifluoromethylphenyl)-4-(4,5-dicyano-1H-imidazol-2-yl)-3-methyl-1-H-pyrazol.

12. A method according to claim 5, wherein the nitroenamine comprises N-(6-chloro-pyridin-3ylmethyl)-N-ethyl-N-methyl-2-methyl-2-nitrovinylidenediamine.

13. A method according to claim 5, wherein:
the arylpyrazole comprises 5-chloro-1-(2,6-dichloro-4-trifluoromethylphenyl)-4-(4,5-dicyano-1H-imidazol-2-yl)-3-methyl-1-H-pyrazol; and
the nitroenamine comprises N-(6-chloro-pyridin-3ylmethyl)-N-ethyl-N-methyl-2-methyl-2-nitrovinylidenediamine.

14. A method according to claim 13, wherein the arylpyrazole and the nitroenamine are administered in a single dosage form.

15. A method according to claim 13, wherein:
at least a portion of the arylpyrazole is administered in a first dosage form, and
at least a portion of the nitroenamine is administered in a second dosage form that is separate from the first dosage form.

16. A method according to claim 13, wherein the arylpyrazole and the nitroenamine are administered to the animal systemically.

17. A method according to claim 13, wherein the arylpyrazole and the nitroenamine are orally administered to the animal.

18. A method according to claim 13, wherein:
the arylpyrazole is administered in a dose of from 0.5 to 10 mg/kg bodyweight, and
the nitroenamine is administered in a dose of from 1 to 30 mg/kg bodyweight.

19. A kit useful for controlling a parasitic insect and/or acarid infestation, wherein:
the kit comprises:
a compound corresponding in structure to Formula (I): 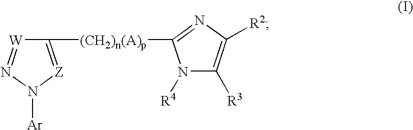
a compound corresponding in structure to Formula (II): 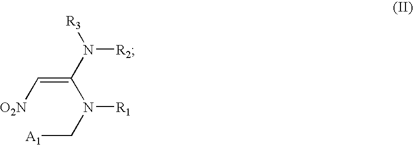
instructions for controlling a parasitic insect and acarid infestation on an animal;
the compounds of Formula (I) and Formula (II) are in a veterinary-acceptable formulation;
Ar is 2,6-dichloro-4-trifluoromethylphenyl or 2-nitro-4-trifluoromethylphenyl;
A is S(O)ₘ, CH═CH, O, or NH;
as to W and Z:
W is N, and Z is CR⁵; or
W is CR¹, and Z is N or CR⁵;
R¹ is hydrogen, optionally substituted alkyl, halogen, or R²⁰S(O)_{q};
R² and R³ are hydrogen, optionally substituted alkyl, optionally substituted alkenyl, optionally substituted alkynyl, aryl, cyano, halogen, nitro, YR²⁰, S(O)₂NR⁸R⁹, CHO, NR⁸R⁹, or CYNR⁸R⁹;
R⁴ is hydrogen, optionally substituted alkyl, optionally substituted alkenyl, acyl, or optionally substituted alkoxycarbonyl, except R⁴ is not alkyl when W is CR¹, Z is CR⁵, and n and p are both zero;
R⁵ is hydrogen, alkyl, optionally substituted amino, or halogen;
R⁸ and R⁹ are:
the same or different, and
are hydrogen, optionally substituted alkyl, acyl, or aryl;
R²⁰ is optionally substituted alkyl;
Y is O or S;
m is zero, 1, or 2;
p is zero or 1;
n is zero, 1, or 2;
q is zero, 1, or 2;
R₁ is hydrogen, C₁-C₆-alkyl, or C₃-C₇-cycloalkyl;
R₂ is hydrogen, C₁-C₆-alkyl, or C₃-C₇-cycloalkyl;
R₃ is hydrogen or C₁-C₆-alkyl;
A₁ is heterocyclyl optionally substituted with one or more identical or different halogens;
unless otherwise stated, any alkyl, alkoxy, and alkylthio has from 1 to 4 carbons;
unless otherwise stated, any alkenyl or alkynyl has from 2 to 5 carbons;
unless otherwise stated, any substituted alkyl, alkoxy, alkylthio, alkenyl, or alkynyl is substituted by one or more of the same or different groups selected from the group consisting of halogen, YR²⁰, dihalocyclopropyl, cyano, nitro, optionally substituted amino, acyloxy, and aryl;
unless otherwise stated, any aryl is phenyl optionally substituted by halogen, alkyl, haloalkyl, alkoxy, haloalkoxy, alkylthio, haloalkylthio, haloalkylsulphonyl, cyano, or nitro;
unless otherwise stated, any acyl is alkanoyl having from 1 to 4 carbons, alkylsulphonyl, or haloalkylsulphonyl; and
unless otherwise stated, any optionally substituted amino corresponds to NR⁸R⁹.

20. A kit according to claim 19, wherein:
the compound of Formula (I) comprises 5-chloro-1-(2,6-dichloro-4-trifluoromethylphenyl)-4-(4,5-dicyano-1H-imidazol-2-yl)-3-methyl-1-H-pyrazol; and
the compound of Formula (II) comprises N-(6-chloro-pyridin-3ylmethyl)-N-ethyl-N-methyl-2-methyl-2-nitrovinylidenediamine.
